SEQUENCE LISTING (4 sequences)

SEQ ID NO: 1 - DNA, 516 bp, Ovis aries
tgctacctgtcgcgaaaactgatgctggacgctcgagaaaatttaaaactgctggaccgtatgaatcgattgtctccgcacagctgcctgcaagaccggaaagacttcggtctgccgcaggaaatggttgaaggtgaccaactgcaaaaagaccaagctttcccggtactgtatgaaatgctgcagcagtctttcaacctgttctacactgaacattcttcggccgcttgggacactactcttctagaacaactgtgcactggtctgcaacagcaactggaccatctggacacttgccgtggccaggttatgggtgaagaagactctgaactgggtaacatggatccgatcgttactgttaaaaaatatttccagggtatctacgactacctgcaggaaaaaggttactctgactgcgcttgggaaatcgtacgcgttgaaatgatgcgggccctgactgtgtcgactactctgcaaaaacggttaactaaaatgggtggtgacctgaattctccg

SEQ ID NO: 2 - protein, 172 aa, Ovis aries
CYLSRKLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGQVMGEEDSELGNMDPIVTVKKYFQGIYDYLQEKGYSDCAWEIVRVEMMRALTVSTTLQKRLTKMGGDLNSP

SEQ ID NO: 3 - protein, 172 aa, Artificial
  Recombinant IFNTau Based on Ovis aries Sequence
CYLSERLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGQVMGEEDSELGNMDPIVTVKKYFQGIYDYLQEKGYSDCAWEIVRVEMMRALTVSTTLQKRLTKMGGDLNSP

SEQ ID NO: 4 - DNA, 516 bp, Artificial
  Recombinant IFNTau Based on Ovis aries Sequence
tgctacctgtcggagcgactgatgctggacgctcgagaaaatttaaaactgctggaccgtatgaatcgattgtctccgcacagctgcctgcaagaccggaaagacttcggtctgccgcaggaaatggttgaaggtgaccaactgcaaaaagaccaagctttcccggtactgtatgaaatgctgcagcagtctttcaacctgttctacactgaacattcttcggccgcttgggacactactcttctagaacaactgtgcactggtctgcaacagcaactggaccatctggacacttgccgtggccaagttatgggtgaagaagactctgaactgggtaacatggatccgatcgttactgttaaaaaatatttccagggtatctacgactacctgcaggaaaaaggttactctgactgcgcttgggaaatcgtacgcgttgaaatgatgcgggccctgactgtgtcgactactctgcaaaaacggttaactaaaatgggtggtgacctgaattctccg

(19) United States
(12) Patent Application Publication
(10) Pub. No.: US 2005/0201981 A1
(43) Pub. Date: Sep. 15, 2005

(54) Method of optimizing treatment with interferon-tau

(76) Inventors: Chih-Ping Liu, San Francisco, CA (US); Lorelie H. Villarete, Alameda, CA (US); Stephen N. Kirnon, San Ramon, CA (US)

Correspondence Address: PERKINS COIE LLP, P.O. BOX 2168, MENLO PARK, CA, 94026, US

(21) Appl. No.: 11/078,608

(22) Filed: Mar. 10, 2005

Related U.S. Application Data
(63) Continuation-in-part of application No. 11/040,706, filed on Jan. 21, 2005.
(63) Continuation-in-part of application No. 10/884,741, filed on Jul. 2, 2004.
(63) Continuation-in-part of application No. 10/825,457, filed on Apr. 14, 2004.
(63) Continuation-in-part of application No. 10/825,382, filed on Apr. 14, 2004.
(63) Continuation-in-part of application No. 10/825,068, filed on Apr. 14, 2004.
(63) Continuation-in-part of application No. 10/824,710, filed on Apr. 14, 2004.
(60) Provisional application No. 60/552,279, filed on Mar. 10, 2004.

(51) Int. Cl. A61K 38/21

(52) U.S. Cl. 424/85.4

(57) ABSTRACT

Improvements in a method of treating a human disease or condition responsive to continued and periodic interferon-tau administration in humans are provided, by adjusting the dose administered to the patient in accordance with the patient's serum IL-10 response.

### CROSS-REFERENCE TO RELATED APPLICATIONS

This application is a continuation-in-part of U.S. patent application Ser. Nos. 11/040,706 filed Jan. 21, 2005; Ser. No. 10/884,741, filed Jul. 2, 2004; Ser. No. 10/825,457 filed Apr. 14, 2004; Ser. No. 10/825,382 filed Apr. 14, 2004; Ser. No. 10/825,068 filed Apr. 14, 2004; and Ser. No. 10/824,710, filed Apr. 14, 2004, all of which claim the benefit of U.S. Provisional Patent Application Ser. No. 60/552,279 filed Mar. 10, 2004.

### FIELD OF THE INVENTION

The present invention relates to methods of optimizing treatment of human diseases or conditions responsive to interferon-tau (IFN-τ) administration in humans.

### BACKGROUND OF THE INVENTION

Interferon-tau (IFN-τ) has been shown to have a wide variety of biological activities. For example, IFN-τ has biological activity as an antiviral agent and an anti-proliferative agent, and in the treatment of autoimmune disorders. Accordingly, IFN-τ has an important role in the treatment of a wide variety of serious diseases, including, for example, autoimmune diseases, such as multiple sclerosis, type I diabetes mellitus, and lupus erythematosus; cell proliferation disorders, including various cancers such as hairy cell leukemia, Kaposis' Sarcoma, chronic myelogenous leukemia, skin cancer, renal cell carcinoma, and ovarian cancer; viral diseases, including hepatitis A, hepatitis B, hepatitis C, HIV infection, HTLV-1, and HTLV-II.

As a treatment agent, IFN-τ has some important advantages over other interferons. Type-I interferons IFN-α and IFN-β as well as type II interferon IFN-γ exhibit significant cytotoxicity. Detrimental toxic effects exerted by these interferons have been observed in clinical trials and patient treatment, and include flu-like symptoms such as fever, chills and lethargy; tachycardia, nausea, weight loss, leucopenia, and neutropenia [Degre, M., Int. J. Cancer 14:699-703 (1974) and Fent, K., and Zbinden, G., Trends Pharm. Sci. 8:100-105 (1987). In contrast, IFN-τ exhibits minimal cytotoxicity both in vitro and in vivo. See, e.g., U.S. Pat. No. 6,060,450.

Furthermore, IFN-τ may be administered orally, for uptake from the GI tract, with good efficacy and efficiency, allowing treatment with the drug through a convenient oral route rather than through a parenteral injection route. This allows for better patient compliance and less patient discomfort. The ability to deliver the compound orally is unexpected in view of the general inability to administer polypeptides orally because of their susceptibility to proteolysis in the GI tract and and/or relatively poor absorption from the gut. Furthermore, the oral route of administration has been found to result in much lower levels of anti-IFN-τ antibodies in the serum of treated laboratory animals, relative to injected IFN-τ. See, e.g., U.S. Pat. No. 6,372,206.

In light of the desirable features of IFN-τ, it is desirable to optimize the treatment response in a patient; in particular, it is desirable to ensure the patient is being treated with an effective dose, and preferably a dose that is optimal or near-optimal in its effectiveness.

### SUMMARY OF THE INVENTION

It has been discovered that treatment of human subjects having a disease or condition responsive to continued and periodic IFN-τ administration may be optimized by comparing the patient's IL-10 response (change in serum IL-10 relative to a baseline value) over an initial treatment period that allows for monitoring the patient's serum IL-10 levels at a plurality of time points, and adjusting the dose of IFN-τ so as to produce an IL-10 response that is consistent with a positive therapeutic response. Accordingly, improvements in a method of treating a human disease or condition responsive to continued and periodic IFN-τ administration in humans are provided.

In a first aspect of the invention, improvements in a method of treating a human disease or condition responsive to continued and periodic IFN-τ administration in humans are provided. The method includes (a) administering to a human subject having such a disease or condition, at each of a plurality of times points over a given time period, a selected, therapeutically indicated amount of IFN-τ. At each of a plurality of time points during the given time period, the patient's serum IL-10 level is measured to determine a change in IL-10 level in the subject over the initial treatment period. Based on the change in measured blood, e.g., serum levels so determined, relative to a baseline value of serum IL-10 determined for the patient, the dose of IFN-τ is adjusted, if appropriate, to adjust the subject's IL-10 response in the direction of a desired response to continued IFN-τ administration. This desired IL-10 response may be a given percentage increase over the baseline, e.g., a 25%, 50%, or 100% increase in serum IL-10 level, or an average IL-10 response in a group of human patients that have been treated successfully for a given condition by IFN-τ administration.

In various embodiments, the administering includes administering ovine IFN-τ or bovine IFN-τ, and the compound is administered orally, in the initial treatment period, at a daily dose of at least about 10⁷ Units/patient and may be as high as 10⁹ Units/patients or more, corresponding to about 0.1 mg to 10 mg/patient, respectively.

After the initial treatment period, the method may be repeated using a higher or lower dose of IFN-α, with the IL-10 response again being evaluated to determine whether a therapeutic response is being achieved, as evidence by a desired IL-10 response, and if not, how the dose should be adjusted to attempt to achieve the desired IL-10 response. In one embodiment of the invention, the IL-10 response is determined by a ratio of the IL-10 response to the response for serum IFN-γ and/or for serum IL-12 in the subject at a plurality of time points during the given time period.

In still other aspects, the method includes an improvement in a method of treating a human disease or condition responsive to continued and periodic IFN-τ administration in humans, by measuring the patient's IFN-γ response (change in serum IFN-γ) or for IL-12 response (change in serum IL-12) to IFN-τ administration over an initial treatment period, and adjusting the dose of IFN-τ, if appropriate, if appropriate, to adjust the subject's IFN-γ or IL-12 response in the direction of a desired response to continued IFN-τ administration. This desired response may be a given percentage decrease with respect to baseline, e.g., a 25%, 50%, or 100% decrease in serum IFN-γ or for IL-12 level, or an average IFN-γ response or IL-12 response in a group of human patients that have been treated successfully for a given condition by IFN-τ administration. The method is applicable, for example, in the treatment of multiple sclerosis.

These and other objects and features of the invention will become more apparent when the following detailed description of the invention is read in conjunction with the accompanying drawings.

### BRIEF DESCRIPTION OF THE DRAWINGS

FIGS. 1A-1C are graphs showing the IL-10 serum level, in pg/mL, in human patients suffering from multiple sclerosis and treated orally with IFN-τ, as a function of time, in days, for patient groups I, II, and III treated daily with 0.2 mg IFN-τ (FIG. 1A), 0.6 mg IFNα (FIG. 1B), and 1.8 mg IFNτ (FIG. 1C) from days 1-28.

FIG. 1D is a graph showing the mean IL-10 serum level, in pg/mL, for the human patients in each of the test Groups I, II, and III treated daily with 0.2 mg IFN-τ (diamonds, Group I), 0.6 mg IFN-τ (squares, Group II), and 1.8 mg IFN-τ (triangles, Group III) from days 1-28.

FIG. 1E illustrates the area-under-the curve-calculations for determining IL-10 response in accordance with one embodiment of the invention.

FIGS. 2A-2C are graphs showing the IFN-τ serum level, in pg/mL, in human patients suffering from multiple sclerosis and treated orally with IFN-τ, as a function of time, in days, for patient groups I, II, and III treated daily with 0.2 mg IFN-τ (FIG. 2A), 0.6 mg IFN-τ (FIG. 2B), and 1.8 mg IFN-τ (FIG. 2C) from days 1-28.

FIG. 2D is a graph showing the mean IFN-γ serum level, in pg/mL, for the human patients in each of the test Groups I, II, and III treated daily with 0.2 mg IFNτ (diamonds, Group I), 0.6 mg IFN-τ (squares, Group II), and 1.8 mg IFN-τ (triangles, Group III) from days 1-28.

FIGS. 3A-3C are graphs showing the IL-10 serum level, in pg/mL, in human patients suffering from hepatitis C and treated orally with IFN-τ, as a function of time, in days, for the six patients in Test Group I treated daily with 0.33 mg IFN-τ three times daily (FIG. 3A), for the six patients in Test Group II treated daily with 1.0 mg IFN-τ three times daily (FIG. 3B); and for the six patients in Test Group III treated daily with 3 mg IFN-τ three times daily (FIG. 3C).

FIG. 3D is a summary plot for the test Groups I, II, and III in FIGS. 3A-3C, showing the percent increase in serum IL-10 levels as a function of time for test Group I (diamonds, 0.33 mg three times daily), Group II (squares, 1 mg three times daily), and Group III (triangles, 3 mg three times daily).

FIGS. 4A-4C are graphs showing the IFN-γ serum level, in pg/mL, in human patients suffering from hepatitis C and treated orally with IFN-τ, as a function of time, in days, for the six patients in Test Group I treated daily with 0.33 mg IFN-τ three times daily (FIG. 4A), for the six patients in Test Group II treated daily with 1.0 mg IFN-τ three times daily (FIG. 4B); and for the six patients in Test Group IIII treated daily with 3 mg IFN-τ three times daily (FIG. 4C).

FIG. 4D is a summary plot for the test Groups I, II, and III in FIGS. 4A-4C, showing the mean serum IFN-γ levels as a function of time for test Group I (diamonds, 0.33 mg three times daily), Group II (circles, 1 mg three times daily), and Group III (triangles, 3 mg three times daily).

FIGS. 5A-5B are graphs showing the IL-10 serum level (FIG. 5A) and the IFN-γ serum level (FIG. 5B), in pg/mL, in human patients suffering from hepatitis C and treated orally with IFN-τ, as a function of time, in days, where a 7.5 mg dose of IFNτ was given twice a day on an empty stomach.

FIGS. 6A-6D show the IL-10 (diamonds), IFN-γ (squares), and IL-12 (triangles) serum levels, in pg/mL, for the six patients treated as described with respect to FIGS. 5A-5B.

### BRIEF DESCRIPTION OF THE SEQUENCES

SEQ ID NO:1 is the nucleotide sequence of a synthetic gene encoding ovine interferon-τ (IFNτ).

SEQ ID NO:2 corresponds to an amino acid sequence of mature ovine interferon-τ (IFNτ; oTP-1; GenBank Accession No. Y00287; PID g1358).

SEQ ID NO:3 corresponds to an amino acid sequence of mature ovine IFNτ, where the amino acid residues at positions 5 and 6 of the sequence are modified relative to the sequence of SEQ ID NO:2.

SEQ ID NO:4 is a synthetic nucleotide sequence encoding the protein of SEQ ID NO:3.

### DETAILED DESCRIPTION OF THE INVENTION

I. Definitions

Unless indicated otherwise, the terms below have the following meaning herein:

“Interferon-tau”, abbreviated as interferon-τ or IFN-τ, refers to any one of a family of interferon proteins having at least one characteristic from each of the following two groups of characteristics: (i) (a) anti-luteolytic properties, (b) anti-viral properties, (c) anti-cellular proliferation properties; and (ii) about 45 to 68% amino acid homology with α-interferons and greater than 70% amino acid homology to known IFNτ sequences (e.g., Ott, et al., J. Interferon Res., 11:357 (1991); Helmer, et al., J. Reprod. Fert., 79:83 (1987); Imakawa, et al., Mol. Endocrinol, 3:127 (1989); Whaley, et al., J. Biol. Chem., 269:10846 (1994); Bazer, et al., WO 94/10313 (1994)). Amino acid homology can be determined using, for example, the LALIGN program with default parameters. This program is found in the FASTA version 1.7 suite of sequence comparison programs (Pearson and Lipman, PNAS, 85:2444 (1988); Pearson, Methods in Enzymology, 183:63 (1990); program available from William R. Pearson, Department of Biological Chemistry, Box 440, Jordan Hall, Charlottesville, Va.). IFNτ sequences have been identified in various ruminant species, including but not limited to, cow (Bovine sp., Helmer S. D., J. Reprod. Fert., 79:83 (1987); Imakawa, K., Mol. Endocrinol., 119:532 (1988)), sheep (Ovine sp.), musk ox (Ovibos sp.), giraffe (Giraffa sp., GenBank Accession no. U55050), horse (Equus caballus), zebra (Equus burchelli, GenBank Accession no. NC005027), hippopotamus (Hippopotamus sp.), elephant (Loxodonta sp.), llama (Llama glama), goat (Capra sp., GenBank Accession nos. AY357336, AY357335, AY347334, AY357333, AY357332, AY357331, AY357330, AY357329, AY357328, AY357327), and deer (Cervidae sp.). The nucleotide sequences of IFNτ for many of these species are reported in public databases and/or in the literature (see, for example, Roberts, R. M. et al., J. Interferon and Cytokine Res., 18:805 (1998), Leaman D. W. et al., J. Interferon Res., 12:1 (1993), Ryan, A. M. et al., Anim. Genet., 34:9 (1996)). The term “interferon-tau” intends to encompass the IFN-τ protein from any ruminant species, exemplified by those recited above, that has at least one characteristic from each of the following two groups of characteristics listed above.

“Ovine IFN-τ (Ov IFN-τ) refers to a protein having the amino acid sequence as identified herein as SEQ ID NO:2, and to proteins having amino acid substitutions and alterations such as neutral amino acid substitutions that do not significantly affect the activity of the protein, such as the IFN-τ protein identified herein as SEQ ID NO:3. More generally, an ovine IFN-τ protein is one having about 80%, more preferably 90%, sequence homology to the sequence identified as SEQ ID NO:2. Sequence homology is determined, for example, by a strict amino acid comparison or using one of the many programs commercially available.

“Treating” a condition refers to administering a therapeutic substance effective to reduce the symptoms of the condition and/or lessen the severity of the condition.

“Oral” refers to any route that involves administration by the mouth or direct administration into the stomach or intestines, including gastric administration.

“Intestine” or “gastrointestinal tract” refers to the portion of the digestive tract that extends from the lower opening of the stomach to the anus, composed of the small intestine (duodenum, jejunum, and ileum) and the large intestine (ascending colon, transverse colon, descending colon, sigmoid colon, and rectum).

A “dosage of greater than about 7×10⁶ Units/kg body weight” refers to an amount of IFN-τ sufficient to provide more than about 5×10⁸ antiviral Units of protein to a person of more than 70 kg weight, where the antiviral activity of IFN-τ is measured using a standard cytopathic effect inhibition assay, such as that described in the Methods section herein. It will be appreciated that the amount (i.e., mg) of protein to provide a daily dosage of greater than 5×10⁸ Units will vary according to the specific antiviral activity of the protein. A reasonable specific antiviral activity for IFN-τ is about 1×10⁸ antiviral units/mg purified protein.

A “therapeutically-indicated amount” of IFN-τ is that amount of IFN-τ which is expected to achieve a desired clinical endpoint, based, for example, on the response shown by other patients treated for the same condition with IFN-τ.

A “desired response to continued interferon-tau administration” refers to an IL-10 response that indicates that the patient is receiving a dose level of IFN-τ that is expected to produce a therapeutically effective results. The desired response may be a given percentage increase over the patient's baseline IL-10 value, e.g., a 25%, 50%, or 100% increase in serum IL-10 level in response to treatment, or an average IL-10 response in a group of human patients that have been treated successfully for a given condition by IFN-τ administration.

“Serum” IL-10 or IL-12 or IFN-γ refers to the level of IL-10 or IL-12 or IFN-γ, respectively, typically expressed as cytokine units/mL, or pg/ml, measured in a blood-derived fraction, typically a serum fraction, but which may include other blood-derived fractions, such as whole blood or plasma.

II. IFN-τ Compositions for the Treatment Method

Therapeutic composition containing IFN-τ, e.g., ovine IFN-τ, are prepared according to published methods. The 172 amino acid sequence of ovine-IFN-τ is set forth, for example, in U.S. Pat. No. 5,958,402, and its homologous bovine-IFNτ sequence is described, for example, in Helmer et al., J. Reprod. Fert., 79:83-91 (1987) and Imakawa, K. et al., Mol. Endocrinol., 3:127 (1989). The sequences of ovine-IFNτ and bovine-IFNτ from these references are hereby incorporated by reference. An amino acid sequence of ovine IFNτ is shown herein as SEQ ID NO:2.

A. Preparation and Properties of IFN-τ

IFN-τ, e.g., ovine IFN-τ, may be prepared for example, as a low molecular weight protein released into conceptus culture medium was purified and shown to be both heat labile and susceptible to proteases (Godkin, J. D., et al., J. Reprod. Fertil. 65:141-150 (1982)).

Alternatively, the protein can be made prepared by recombinant means, using as the coding sequence, an IFN-τ cDNA obtained by probing a sheep blastocyst library with a synthetic oligonucleotide representing the N-terminal amino acid sequence (Imakawa, K. et al, Nature, 330:377-379, (1987)). Several cDNA sequences have been reported which may represent different isoforms (Stewart, H. J., et al, Mol. Endocrinol. 2:65 (1989); Klemann, S. W., et al., Nuc. Acids Res. 18:6724 (1990); and Charlier, M., et al., Mol. Cell Endocrinol. 76:161-171 (1991)). All are approximately 1 kb with a 585 base open reading frame that codes for a 23 amino acid leader sequence and a 172 amino acid mature protein. The predicted structure of IFNτ as a four helical bundle with the amino and carboxyl-termini in apposition further supports its classification as a type I IFN (Jarpe, M. A., et al., Protein Engineering 7:863-867 (1994)). Methods for producing recombinant ovine IFN-τ are given in the Methods section of the Examples below.

While IFN-τ displays some of the activities classically associated with type I IFNs (see Table 1 below), considerable differences exist between it and the other type I IFNs. The most prominent difference is its role in pregnancy, detailed above. Also different is viral induction. All type-I IFNs, except IFNτ, are induced readily by virus and dsRNA (Roberts, R. M., et al., Endocrin. Rev. 13:432-452 (1992)). Induced IFN-α and IFN-β expression is transient, lasting approximately a few hours. In contrast, IFNτ synthesis, once induced, is maintained over a period of days (Godkin, et al., 1982). On a per-cell basis, 300-fold more IFNτ is produced than other type I IFNs (Cross, J. C., and Roberts, R. M., Proc. Natl. Acad. Sci. USA 88:3817-3821 (1991)).

TABLE 1
Overview of the Interferons

| | Aspects | | | |
|---|---|---|---|---|
| | Type I | Type I | Type I | Type II |
| | Types | | | |
| | α & ω | β | τ | γ |
| | Produced by: | | | |
| | Leukocyte | fibroblast | trophoblast | lymphocyte |
| Antiviral | + | + | + | + |
| Antiproliferative | + | + | + | + |
| Pregnancy Signaling | − | − | + | − |

B. IFN-τ Formulations

Oral or parenteral preparations containing IFN-τ can be formulated according to known methods for preparing pharmaceutical compositions. In general, the IFN-τ therapeutic compositions are formulated such that an effective amount of the IFN-τ is combined with a suitable additive, carrier and/or excipient in order to facilitate effective oral administration of the composition. For example, tablets and capsules containing IFN-τ may be prepared by combining IFN-τ (e.g., lyophilized IFN-τ protein) with additives such as pharmaceutically acceptable carriers (e.g., lactose, corn starch, microcrystalline cellulose, sucrose), binders (e.g., alpha-form starch, methylcellulose, carboxymethyl cellulose, hydroxypropyl cellulose, hydroxypropyl methylcellulose, polyvinylpyrrolidone), disintegrating agents (e.g., carboxymethylcellulose calcium, starch, low substituted hydroxy-propylcellulose), surfactants (e.g., Tween 80, polyoxyethylene-polyoxypropylene copolymer), antioxidants (e.g., L-cysteine, sodium sulfite, sodium ascorbate), lubricants (e.g., magnesium stearate, talc), or the like.

Further, IFN-τ polypeptides of the present invention can be mixed with a solid, pulverulent or other carrier, for example lactose, saccharose, sorbitol, mannitol, starch, such as potato starch, corn starch, millopectine, cellulose derivative or gelatine, and may also include lubricants, such as magnesium or calcium stearate, or polyethylene glycol waxes compressed to the formation of tablets. By using several layers of the carrier or diluent, tablets operating with slow release can be prepared.

Liquid preparations for oral administration can be made in the form of elixirs, syrups or suspensions, for example solutions containing from about 0.1% to about 30% by weight of IFN-τ, sugar and a mixture of ethanol, water, glycerol, propylene, glycol and possibly other additives of a conventional nature.

Another suitable formulation is a protective dosage form that protects the protein for survival in the stomach and intestines until absorbed by the intestinal mucosa. Protective dosage forms for proteins are known in the art, and include enteric coatings and/or mucoadhesive polymer coatings. Exemplary mucoadhesive polymer formulations include ethyl cellulose, hydroxypropylmethylcellulose, Eudragit®, carboxyvinly polymer, carbomer, and the like. A dosage form designed for administration to the stomach via ingestion for delivery of IFN-τ in an active form to the intestinal tract, and particularly to the small intestine, is contemplated. Alternatively, IFN-τ can be co-administered with protease inhibitors, stabilized with polymeric materials, or encapsulated in a lipid or polymer particle to offer some protection from the stomach and/or intestinal environment.

III. Treatment Method

The present method is an improvement in a therapeutic method for treating a patient that is responsive to treatment with IFN-τ administration, e.g., by oral administration of IFN-τ to the patient. The improvement is based on the discovery that the patient's response to IFN-τ administration can be monitored by the change in the patient's serum IL-10 levels in response to the treatment. If the patient shows a desired serum IL-10 response during an initial treatment period, the original dose may be maintained as a therapeutically effective one. If the response is low, the IFN-τ dose may be increased until a desired IL-10 response is achieved. In some case, particularly where the original treatment dose is one sufficient to cause a desired IL-10 response in most human patients with the particular condition being treated, the physician may decrease the dose in an effort to improve the IL-10 response of the patient.

The method can also involve monitoring the patient's serum IL-12 of IFN-γ levels during the initial treatment period, to determine a patient's IL-12 of IFN-γ response. Both cytokines have been shown, in accordance with the present invention, to be responsive to IFN-τ administration, typically decreasing over the treatment period, at least in some disease states such as multiple sclerosis. The measured IL-12 of IFN-γ response can be employed either independently as a measure of patient responsiveness to IFN-τ administration, or may be used to refine the IL-10 response, typically by expressing IL-10 response as a ratio of IL-10 response/IL-12 response or IL-10 response/IFN-γ response.

Where no statistically significant increased IL-10 response is observed in response to IFN-τ administration, particularly at higher doses of the compound, and where no statistically significant decreased IL-12 or IFN-γ response is observed, the physician may conclude that the patient should be placed an alternative therapy or a therapy which includes IFN-τ and an additional treatment agent.

A. Basic Treatment Method

The patients or individuals for whom the treatment method is intended are those having a disease conditions that is responsive to continued and period IFN-τ administration. A disease or condition “responsive to IFN-τ administration” is one in which the existence, progression, or symptoms of the condition is altered upon administration of IFN-τ. The method described herein encompasses providing IFN-τ, preferably in an orally-administrable dosage form for administration to the stomach and/or intestines, to subjects that are immune-responsive to IFN-τ treatment as evidenced by an increase in serum IL-10 levels measured at a selected periods after administering IFN-τ, compared to a baseline value of serum IL-10.

IFN-τ has biological activity as, for example, an antiviral agent, an anti-proliferative agent, and in treatment of autoimmune disorders (see for example U.S. Pat. Nos. 5,958,402; 5,942,223; 6,060,450; 6,372,206). Accordingly, examples of disease or conditions responsive to interferon-tau administration include, for example, autoimmune, inflammatory, viral infections, proliferative and hyperproliferative diseases, as well as immunologically-mediated diseases. Below are given specific disease conditions that are responsive to IFN-τ administration, and therefore suitable for the present invention.

A1. Treatment of Immune System Disorders

The method of the present invention is advantageous for treating conditions relating to immune system hypersensitivity. There are four types of immune system hypersensitivity (Clayman, C. B., Ed., AMERICAN MEDICAL ASSOCIATION ENCYCLOPEDIA OF MEDICINE, Random House, New York, N.Y., (1991)). Type I, or immediate/anaphylactic hypersensitivity, is due to mast cell degranulation in response to an allergen (e.g., pollen), and includes asthma, allergic rhinitis (hay fever), urticaria (hives), anaphylactic shock, and other illnesses of an allergic nature. Type II, or autoimmune hypersensitivity, is due to antibodies that are directed against perceived “antigens” on the body's own cells. Type III hypersensitivity is due to the formation of antigen/antibody immune complexes which lodge in various tissues and activate further immune responses, and is responsible for conditions such as serum sickness, allergic alveolitis, and the large swellings that sometimes form after booster vaccinations. Type IV hypersensitivity is due to the release of lymphokines from sensitized T-cells, which results in an inflammatory reaction. Examples include contact dermatitis, the rash of measles, and “allergic” reactions to certain drugs.

The mechanisms by which certain conditions may result in hypersensitivity in some individuals are generally not well understood, but may involve both genetic and extrinsic factors. For example, bacteria, viruses or drugs may play a role in triggering an autoimmune response in an individual who already has a genetic predisposition to the autoimmune disorder. It has been suggested that the incidence of some types of hypersensitivity may be correlated with others. For example, it has been proposed that individuals with certain common allergies are more susceptible to autoimmune disorders.

Autoimmune disorders may be loosely grouped into those primarily restricted to specific organs or tissues and those that affect the entire body. Examples of organ-specific disorders (with the organ affected) include multiple sclerosis (myelin coating on nerve processes), type I diabetes mellitus (pancreas), Hashimotos thyroiditis (thyroid gland), pernicious anemia (stomach), Addison's disease (adrenal glands), myasthenia gravis (acetylcholine receptors at neuromuscular junction), rheumatoid arthritis (joint lining), uveitis (eye), psoriasis (skin), Guillain-Barré Syndrome (nerve cells) and Grave's disease (thyroid). Systemic autoimmune diseases include systemic lupus erythematosus and dermatomyositis. Another autoimmune disorder is Sjogren's syndrome, where white blood cells attack the moisture-producing glands. The hallmark symptoms of Sjogren's syndrome are dry eyes and dry mouth, but it is a systemic disease, affecting many organs.

Other examples of hypersensitivity disorders include asthma, eczema, acne, atopical dermatitis, contact dermatitis, other eczematous dermatitis, seborrheic dermatitis, rhinitis, Lichen planus, Pemplugus, bullous Pemphigoid, Epidermolysis bullosa, uritcaris, angioedemas, vasculitides, erythemas, cutaneous eosinophilias, Alopecia greata, atherosclerosis, primary biliary cirrhosis and nephrotic syndrome. Related diseases include intestinal inflammations, such as Coeliac disease, proctitis, eosinophilia gastroenteritis, mastocytosis, inflammatory bowel disease, Crohn's disease and ulcerative colitis, as well as food-related allergies. Ankylosing spondylitis is another example of an autoimmune, inflammatory disease, where some or all of the joints and bones of the spine fuse together.

Optimization of treatment with IFN-τ according to the methods of the present invention is particularly advantageous for subjects with autoimmune diseases such as multiple sclerosis, type I (insulin dependent) diabetes mellitus, lupus erythematosus, amyotrophic lateral sclerosis, Crohn's disease, rheumatoid arthritis, stomatitis, asthma, uveitis, allergies, psoriasis, Ankylosing spondylitis, Myasthenia Gravis, Grave's disease, Hashimoto's thyroiditis, Sjogren's syndrome, and inflammatory bowel disease.

A2. Viral Infections

The methods of the invention may be applied to optimize treatment of diseases or conditions associated with viral infection. The antiviral activity of IFN-τ has broad therapeutic applications without the toxic effects that are usually associated with IFN-αs, and IFN-τ exerts its therapeutic activity without adverse effects on the cells. The relative lack of cytotoxicity of IFN-τ makes it extremely valuable as an in vivo therapeutic agent and sets IFN-τ apart from most other known antiviral agents and all other known interferons.

The viral infection can be due to a RNA virus or a DNA virus. Examples of specific viral diseases which may be treated by orally-administered IFN-τ include, but are not limited to, hepatitis A, hepatitis B, hepatitis C, non-A, non-B, non-C hepatitis, Epstein-Barr viral infection, HIV infection, herpes virus (EB, CML, herpes simplex), papilloma, poxvirus, picoma virus, adeno virus, rhino virus, HTLV I, HTLV II, and human rotavirus.

A3. Cell Proliferation Disorders

IFN-τ exhibits potent anticellular proliferation activity. Accordingly, the methods of the present invention may be applied to optimize treatment of subjects to inhibit, prevent, or slow uncontrolled cell growth.

Examples of cell proliferation disorders in humans which may be treated by orally-administered IFN-τ include, but are not limited to, lung large cell carcinoma, colon adenocarcinoma, skin cancer (basal cell carcinoma and malignant melanoma), renal adenocarcinoma, promyelocytic leukemia, T cell lymphoma, cutaneous T cell lymphoma, breast adenocarcinoma, steroid sensitive tumors, hairy cell leukemia, Kaposi's Sarcoma, chronic myelogenous leukemia, multiple myeloma, superficial bladder cancer, ovarian cancer, and glioma.

A4. Other Disorders

Further examples of diseases or conditions responsive to interferon-tau administration include, for example, neurological diseases, such as Alzheimer's disease and autism; fibrotic diseases, including pulmonary fibrosis and liver fibrosis; stroke, rejection from organ transplantation; and chronic obstructive pulmonary disease, including chronic bronchitis and emphysema.

B. Initial and Adjusted Treatment Doses

At the beginning of the treatment, the patient's baseline level of serum IL-10 and optionally, serum IL-12 and/or serum IFN-γ baseline values are determined. This may be done, as in the examples below, by taking a single blood measurement before (or concurrently with) the initial IFN-τ administration, i.e., when a patient is in an uninduced state. Alternatively, the baseline value(s) may be determined by taking a number of blood measurements over a several day period prior to the beginning of treatment to establish a level of variation in the measured cytokine prior to treatment. These values are then used to establish a baseline value, for example, using the area-under-the curve approach described in Example 1. Actual blood measurements may be made with commercially available immunoassay or other diagnostic kits for assaying IL-10, IL-12 and IFN-γ, as noted in the examples.

With a baseline value of IL-10 and optionally, IL-12 and IFN-γ, established, the patient is placed on an initial treatment at a selected level of IFN-τ, typically given in oral form. Typical initial doses may range from about 0.1 mg/daily to up to 10 mg/daily or higher (corresponding to about 1.4×10⁵ Units/kg body weight and 1.4×10⁷ Units/kg body weight, respectively). This dose is referred to herein as a therapeutically indicated dose and represents an initial-treatment dose that may be an arbitrarily chosen dose, e.g., in the mid-range of doses noted above, or one that corresponds to doses that have been shown in at least some patients, to be therapeutically effective in treating the disease being addressed.

This initial dose is administered over an initial treatment period of typically 2-4 weeks, during which the patient's IL-10 response (and/or IL-12 or IFN-γ response) is monitored. Based on the response measured, the dose of the compound is either maintained, increased, or decreased to adjust the IL-10 response in the direction of a desired response. Details of the dose adjustment, based on the IL-10 response, are discussed in Section B below, with reference particularly to Examples 2-4. Details of dose adjustment, based on additional IL-12 or IFN-γ responses, are discussed in Section C.

B1. Adjusting Treatment Dose on the Basis of IL-10 Response

IL-10 is involved in regulating the functions of various immune system cells, including lymphoid and myeloid cells. IL-10 is a potent suppressor of the effector functions of, for example, macrophages, T cells and natural killer cells and is thought to act through blocking activation of cytokine synthesis and several accessory cell functions of macrophages [Moore, K. W., et al., Annu Rev Immunol. 11:165-90 (1993)]. It has been shown or suggested that a wide variety of diseases or conditions may be benefited by interleukin-10 therapy or are otherwise linked to IL-10. Examples of such diseases or conditions include, for example, neurological diseases, immunological diseases, autoimmune diseases, and viral diseases.

After administering IFN-τ, such as by an oral route, and including administration to the intestinal tract of the subject, a blood sample may be drawn and the serum IL-10 level may then be measured in the subject according to the methods of the present invention at selected time points after administering. For example, the serum IL-10 level may be measured 1, 2, 3, 4 or 5 days after the initial administration or may be measured on a daily basis after the initial administration. Where multiple administrations are provided, serum IL-10 may similarly be measured at various time points, including after each administration, or on a daily or weekly basis. Methods for measuring serum IL-10 levels include the use of commercially available enzyme-linked immunosorbent assay (ELISA) kits.

The serum IL-10 level measured after administration of IFN-τ is then compared with an uninduced serum IL-10 level in the subject to determine the patient's IL-10 response to the initial treatment dose. By “uninduced serum IL-10 level” it is meant the level of serum IL-10 prior to induction of serum IL-10 in the subject by or through IFN-τ administration. The uninduced level may be determined from a single blood sample taken prior to or substantially concurrent with the beginning of treatment, as illustrated in the examples below, or may be determined from a number of blood samples taken at various time points, e.g., every few days, in a time period prior to treatment.

The uninduced serum IL-10 level may vary, but may be between about 1 pg/mL to about 5 pg/mL. The induced serum IL-10 level is typically no greater than about 10 pg/mL or about 20 pg/mL. As will be see from the data in the examples, the patient's response to treatment will typically raise IL-10 levels, with an increase of at least 25% generally indicating a statistically meaningful increase in IL-10 levels. Depending on the disease state, and the dose of IFN-τ being administered, an IL-10 response of 25%-50% is desired, and may be as high as 100% or more. Alternatively, a desired IL-10 response may be determined by the IL-10 responses observed in a group of human patients who have been successfully treated for a given condition with IFN-τ. That is, for a group of patients who have been successfully treated at a given dose, the desired IL-10 response may be the average IL-10 response of this group.

Once the patient's IL-10 response for the initial treatment period has been determined, the dose of IFN-τ may be maintained, increased, or decreased to adjust the subject's IL-10 response in the direction of a desired response to continued to IFN-τ administration. This adjustment, in turn, may be monitored during a second, similar-length treatment period, with the IL-10 response being determined over this second period. Based on the second IL-10 response, the dose of IFN-τ administered may again be adjusted to achieve a desired IL-10 response end point, or may be maintained.

During any of these initial treatment periods, IFN-τ is administered typically once, twice or three times per day for a period of, for example, 2 to 4 weeks, over which serum IL-10 levels are monitored several times, e.g., every 3-4 days. Once a desired IL-10 response is achieved, the treatment may be continued at the last dose level until the disease condition is resolved or as needed to control the course of the disease.

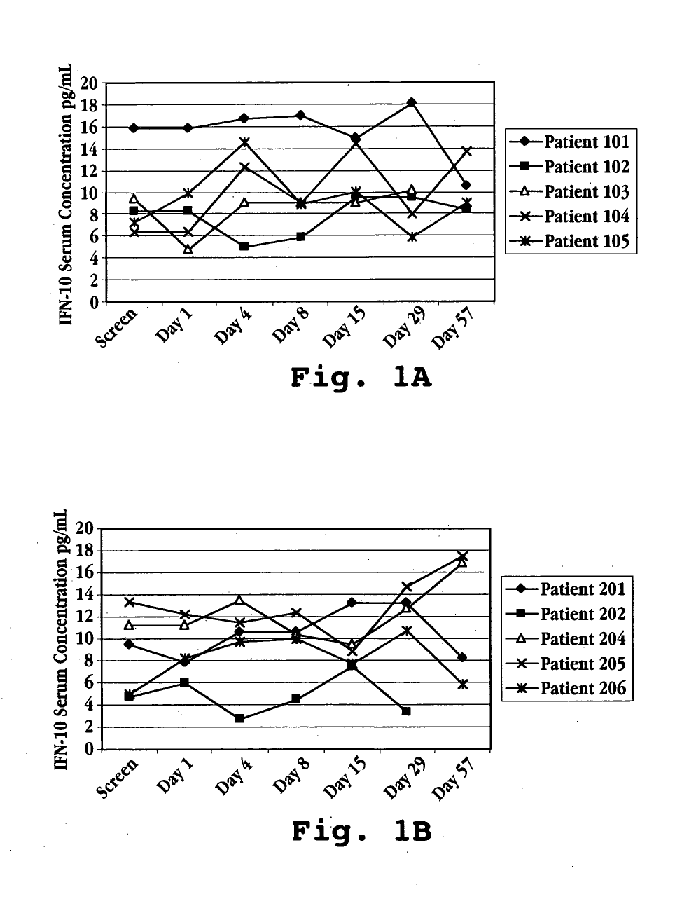

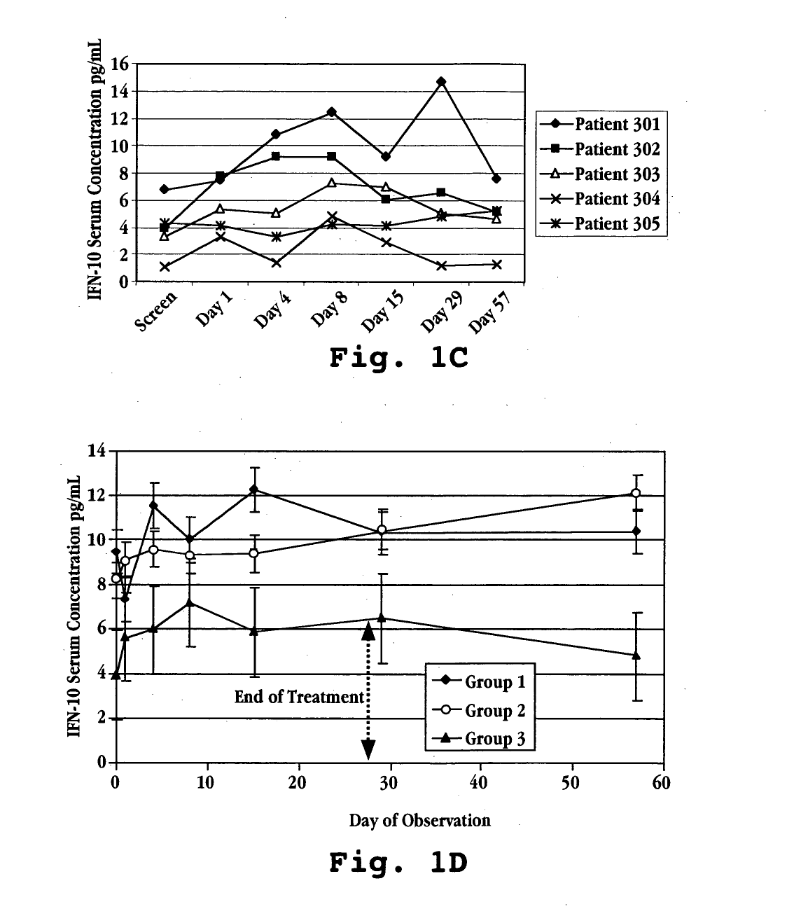

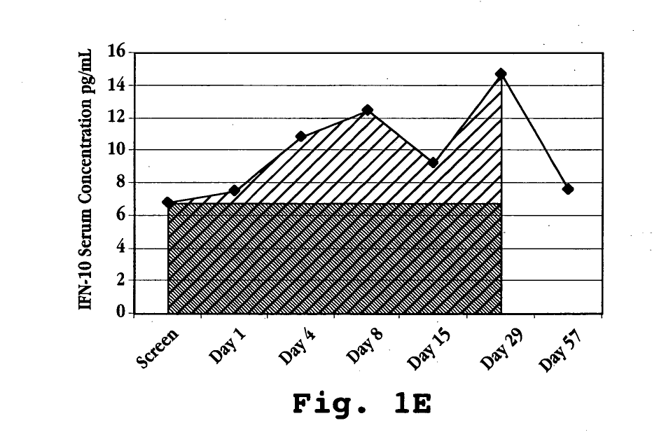

Example 2 illustrates the method as applied to groups of patients being treated with IFN-τ for control of multiple sclerosis. Three groups of five patients each were each given a selected daily dose of 0.2 (Group I), 0.6 (Group II), and 1.8 mg (Group III)/per patient, over a 28 day initial treatment period, as shown in Table 2. (1 mg IFN-τ is approximately 1×10⁸ antiviral Units). Serum IL-10 was monitored at Days, 1, 4, 8, 15, and 29, with the levels shown for the three groups in FIGS. 1A, 1B, and 1C, respectively. The IL-10 response of each patient was determined by an area-under the curve calculation described in Example 1, with the results shown in Table 3 of Example 2.

TABLE 2
MS Patient Group

| | Group I | Group II | Group III |
|---|---|---|---|
| | (n = 5) | (n = 5) | (n = 5) |
| IFN-τ Oral | 0.2 mg/day | 0.6 mg/day | 1.8 mg/day |
| Dose¹ | (2 × 10⁷ U) | (6 × 10⁷ U) | (1.8 × 10⁸ U) |
| Average | 67.2 kg | 58.9 kg | 90.0 kg |
| Weight | | | |
| Average Age | 39 | 34.5 | 47 |

¹1 mg IFN-τ = 1 × 10⁸ Units

The results from Table 3 indicate that the most favorable initial treatment dose is 1.8 mg/day, which led to an IL-10 response of about 1.5 or higher in four of the five patients, but in only one patient each at the lower two doses. Therefore, reducing the dose from this level would not be expected to enhance the IL-10 response, and if a patient receiving an initial 1.8 mg/daily dose does not show an IL-10 response of at least about 1.5, the dose should be increased, not decreased in an effort to boost the IL-10 response.

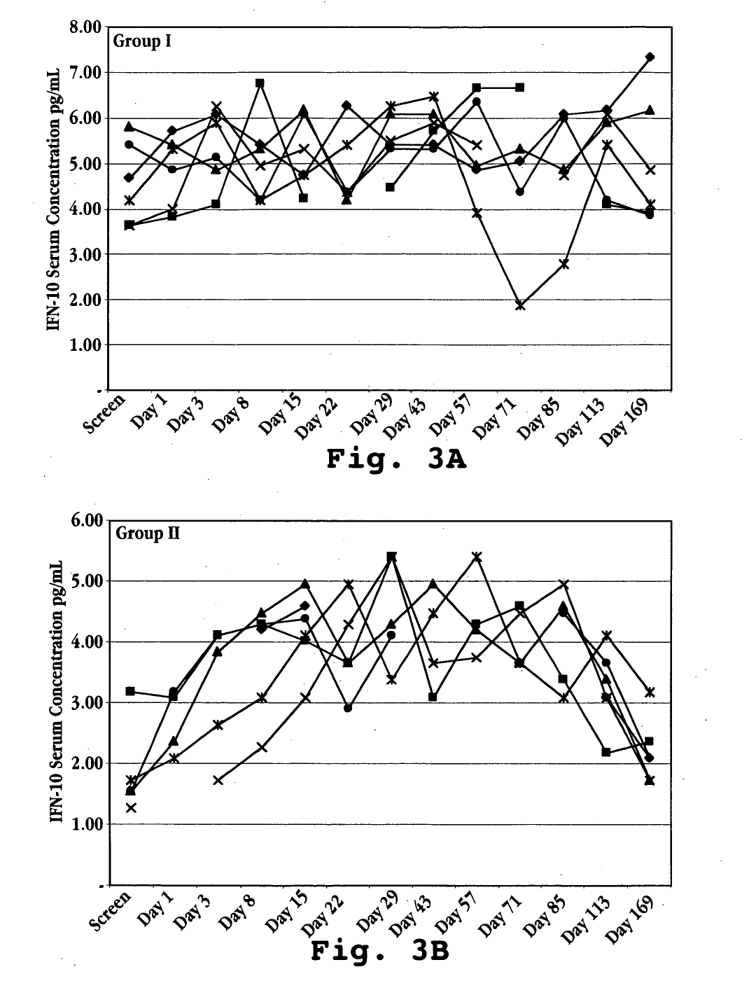

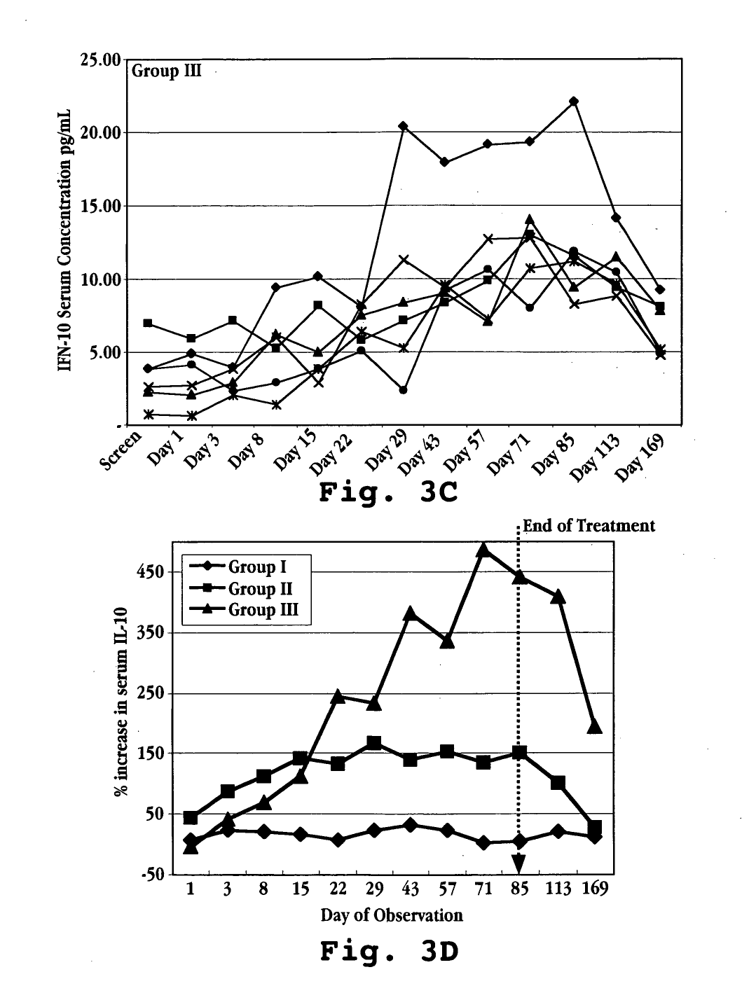

In a second study, detailed in Example 3, patients with HCV were treated divided into three groups by and were administered daily doses of IFN-τ totaling 1 mg (Group I), 3 mg (Group II), and 9 mg (Group-III) daily, where the doses were administered three time daily, i.e., at individual doses of 0.33, 1, and 3 mg, and over a 84 day period. Serum IL-10 levels were monitored at Days 1, 3, 8, 15, 22, 29, 43, 71, and 85, and plotted in FIGS. 3A-3C. The IL-10 response in each group was calculated for the first 28 days of treatment, as in Example 1, with the results shown in Table 7 in Example 3.

As can be appreciated from the IL-10 response data in Table 3, an IL-10 response of at least about 2, i.e., a 100% increase, can be expected in human HCV patients receiving either 3 mg/day or 9 mgs/day IFN-τ, seen in four of the six patients in each group, and in none of the lowest-dose group (1 mg/day). Thus, the 3 mg/day dose would be a good initial dose, consistent with the data above for MS patients which indicated a preferred starting dose of 1.8 mg/day. If the patient shows a strong IL-10 response, e.g., at least a 100% increase in IL-10 over baseline, the dose could either be maintained over extended treatment, or even reduced somewhat to determine whether the IL-10 response is seen at a somewhat lower dose, e.g., 2 mg/day. The failure of some patients to achieve a good IL-10 response even at the highest doses may be an indication that those patients are less responsive to IFN-τ treatment, and might therefore be placed on a second anti-viral treatment agent or alternatively switched to another therapy, particularly if no significant change in IL-12 or IFN-γ is observed over the same treatment period.

C. INF-γ Response

IFN-γ is a pro-inflammatory cytokine, and up-regulation of IFN-γ is correlated with increased discomfort in patients suffering from autoimmune conditions, such as multiple sclerosis and arthritis. During treatment of, for example, multiple sclerosis with interferon-beta (IFN-β), it has been reported that the frequency of IFN-γ-secreting cells increases during the first two months of IFN-β treatment, and this increase of IFN-γ serum levels possibly contributes to the prominent “flu-like” symptoms that patients experience during treatment with IFN-β. One advantage of IFN-τ over treatment with other Type-I interferons, is substantially reduced side effects, and this is reflected in IFN-γ blood levels that is actually lower, in the treatment of certain disease states, than baseline values during IFN-τ treatment.

Accordingly, the present method contemplates using a patient's IFN-γ response, either alone or in calculating an IL-10 response, to determine the patient's response to IFN-τ treatment. Where the response of IFN-γ is used in the determination of IL-10 response, the IL-10 response may be determined as a ratio of IL-10 response/IFN-γ response, as discussed in Examples 2 and 3. As noted in these examples, a patient's blood IFN-γ levels are likely to fall in response to IFN-τ treatment, particularly for certain disease states like multiple sclerosis.

D. IL-12 Response

IL-12 is a pro-inflammatory cytokine and is reported to contribute to, for example, the pathogenesis of multiple sclerosis (MS; Filson et al., Clin. Immunol., 106(2):127 (2003). It is further known that MS patients typically display decreased IL-10 and increased IL-12 levels, and the levels of these cytokines correlate with the disease stage [van Boxel-Dezaire et al., Ann. Neurol., 45:695 (1999)]. With respect to viral infections, a high IL-12 level has also been shown to exacerbate bacterial colonization of B. pertussis [Carter et al., Clin. Exp. Immunol., 135(2):233 (2004)].

As with IFN-γ response, it has been found in accordance with the invention that IL-12 levels can decrease during IFN-τ treatment, providing still another cytokine response that can be used to monitor a patient's response to IFN-τ treatment. As above, the IL-12 response can be used alone, or in combination with IL-10, where the IL-10 response is calculated as a ratio of IL-10 response/IL-12 response.

Although the invention has been described with reference to particular methods and embodiment, it will be appreciated that various modification and variations can be made without departing from the claimed method.

### IV. EXAMPLES

Reference will now be made to specific examples illustrating the invention described above. It is to be understood that the examples are provided to illustrate preferred embodiments and that no limitation to the scope of the invention is intended thereby. Additionally, all documents cited herein are indicative of the level of skill in the art and are hereby incorporated by reference in their entirety.

Materials and Methods

A. Production of IFNτ

In one embodiment, a synthetic IFNτ gene was generated using standard molecular methods (Ausubel, et al., supra, 1988) by ligating oligonucleotides containing contiguous portions of a DNA sequence encoding the IFNτ amino acid sequence. The DNA sequence used may be either SEQ ID NO:1 or SEQ ID NO:4 or the sequence as shown in Imakawa, K. et al, Nature, 330:377-379, (1987). The resulting IFNτ polynucleotide coding sequence may span position 16 through 531: a coding sequence of 172 amino acids.

In one embodiment, the full length synthetic gene StuI/SStI fragment (540 bp) may be cloned into a modified pIN III omp-A expression vector and transformed into a competent SB221 strain of E. coli. For expression of the IFNτ protein, cells carrying the expression vector were grown in L-broth containing ampicillin to an OD (550 nm) of 0.1-1, induced with IPTG (isopropyl-1-thio-b-D-galactoside) for 3 hours and harvested by centrifugation: Soluble recombinant IFN-τ may be liberated from the cells by sonication or osmotic fractionation.

For expression in yeast, the IFNτ gene may amplified using polymerase chain reaction (PCR; Mullis, K. B., U.S. Pat. No. 4,683,202, issued 28 Jul. 1987; Mullis, K. B., et al., U.S. Pat. No. 4,683,195, issued 28 Jul. 1987) with PCR primers containing StuI and SacI restriction sites at the 5′ and 3′ ends, respectively. The amplified fragments were digested with StuI and SacII and ligated into the SacI and SmaI sites of pBLUESCRIPT+(KS), generating pBSY-IFNτ. Plasmid pBSY-IFNτ was digested with SacII and EcoRV and the fragment containing the synthetic IFNτ gene was isolated. The yeast expression vector pBS24Ub (Ecker, D. J., et al., J. Biol. Chem. 264:7715-7719 (1989)) was digested with SalI. Blunt ends were generated using T4 DNA polymerase. The vector DNA was extracted with phenol and ethanol precipitated (Sambrook, J., et al., in MOLECULAR CLONING: A LABORATORY MANUAL, Second Edition, Cold Spring Harbor Laboratory, Cold Spring Harbor, N.Y. (1989)). The recovered plasmid was digested with SacII, purified by agarose gel electrophoresis, and ligated to the SacII-EcoRV fragment isolated from pBSY-IFNτ. The resulting recombinant plasmid was designated pBS24Ub-IFNτ.

The recombinant plasmid pBS24Ub-IFNτ was transformed into E. coli. Recombinant clones containing the IFNτ insert were isolated and identified by restriction enzyme analysis. IFNτ coding sequences were isolated from pBS24Ub-IFNτ and cloned into a Pichia pastoris vector containing the alcohol oxidase (AOX1) promoter (Invitrogen, San Diego, Calif.). The vector was then used to transform Pichia pastoris GS115 His⁻ host cells and protein was expressed following the manufacturer's instructions. The protein was secreted into the medium and purified by successive DEAE-cellulose and hydroxyapatite chromatography to electrophoretic homogeneity as determined by SDS-PAGE and silver staining.

B. Antiviral Assay to Determine Specific Antiviral Activity

Antiviral activity was assessed using a standard cytopathic effect assay (Familletti, P. C., et al., Methods in Enzymology, 78:387-394 (1981); Rubinstein, S. et al., J. Virol, 37:755-758 (1981)). Briefly, dilutions of IFNτ were incubated with Madin-Darby bovine kidney (MDBK) cells for 16-18 hours at 37° C. Following incubation, inhibition of viral replication was determined in a cytopathic effect assay using vesicular stomatitis virus as challenge. One antiviral unit (U) caused a 50% reduction in destruction of the monolayer. For the studies described herein, the IFNτ had a specific activity of about 1×10⁸ antiviral U/mg protein.

### Example 1

### Determination of Cytokine Levels

This example illustrates one method for determining IL-10 response in a patient. As discussed above, the IL-10 response is a measure of the extent to which serum IL-10 levels have changed in the patient as a result of IFN-τ administration. In the method described in this example, IL-10 response is calculated as the ratio of the area-under-the-curve (AUC) for serum IL-10 levels over an initial treatment period to the level of serum IL-10 that would be expected over the same period in the absence of any treatment, i.e., a baseline IL-10 level.

FIG. 12E illustrates how this value is determined. The IL-10 levels in the figure are taken from an MS patient (patient 302 in FIG. 1C) who is receiving a daily oral dose of 1.8 mg over a period of Day 1 to Day 28. At Day 29, the treatment is discontinued. The AUC for IL-10 response is the total area under the curve defined by the IL-10 measurements in the treated patient. The baseline value may be calculated as the area under the curve defined by an initial value taken at the time treatment is initiated (or before initiation of treatment) and is considered over the same time period of Day 1 to Day 29, and calculated as the area of the resulting rectangle. Alternatively, the baseline value may be calculated as a true area under the curve by taking a number of IL-10 measurements prior to treatment and calculating an AUC value for the baseline value. In the latter case, the baseline AUC and IL-10 AUC have to be corrected to the same sampling period, e.g., 28 days, before taking the ratio of the two AUC's.

For the IL-10 response curve, the AUC may be calculated easily using the Trapezoidal Rule, in which the area to be measured is broken up into multiple trapezoids and the sum of the area of all of the trapezoids is determined and represents the area under the curve. The method is detailed, for example, on the website www.boomer.org/c/P3/c02/c0210.html. In performing these calculations an area under the curve for the post-treatment points in FIG. 1E was obtained (All area under the curve values are in units of pg days/ml.) The baseline AUC was calculated, as above, simply as the area of the baseline rectangle shown. The IL-10 response was then calculated as the ratio of the two values.

In addition, IL-10 response may be calculated as a ratio of IL-10 response to the patient's response in serum levels of IL-12 and/or IFN-γ. In this case, one calculates an IL-12 or IFN-γ response by the same method just described for calculating IL-10 response. Once the two response values are calculated, the IL-10 response is calculated as the ratio of IL-10 response/IL-12 or IL-10/IFN-γ response. In two of the examples below, an IL-10 response expressed as IL-10/IFN-γ is reported.

### Example 2

### Initial-Treatment IL-10 Level Response in MS patients

This example illustrates a typical relationship between initial-treatment dose of IFNτ and IL-10 response measured over an initial treatment period of 28 days. The human patients in this study had multiple sclerosis and were enrolled in a trial for treatment with IFNτ. Fifteen patients were randomized into three treatment groups (see Table 2 above): Group I patients were given IFNτ orally at a dosage of 0.2 mg per day (2×10⁷ U/day) Group II patients were given IFNτ orally at a dosage of 0.8 mg per day (8×10⁷ U/day); and Group III patients were given IFNτ orally at a dosage of 1.8 mg per day (1.8×10⁸ U/day), the patients in each group receiving a once daily dose for 29 days.

Prior to treatment with IFNτ, on screening Day and Day 1 (one), a blood sample was taken from each subject to determine a baseline serum cytokine concentration. Treatment was initiated by administering IFNτ orally to each patient following the blood draw on Day 1. Prior to administration, the vials of IFNτ (SEQ ID NO:3) and syringes were kept in a refrigerator maintained at 2 to 8° C. Prior to self-administration of medication, the patient removed one vial and one syringe from the refrigerator. The cap was removed from the tip of the syringe and the tip of the syringe was placed into the bottle of medication to withdraw the appropriate volume into the syringe as instructed at the clinic on Day 1. The tip of the syringe was placed in the mouth and the syringe contents were emptied into the mouth by depressing the plunger. The patient then swallowed, and if desired, was allowed to drink a glass of water. The patient noted on his/her diary card the date and time the dose was administered.

A. IL-10 Response Measured by IL-10 AUC/Baseline AUC

Blood samples were taken from each patient on Days 1, 4, 8, 15, 29, and 57 of the study. The samples were analyzed for IL-10 concentrations by using commercially available ELISA kits (Genzyme, Cambridge, Mass). The levels of IL-10 were graphically plotted as shown in FIGS. 1A-1C (IL-10) and the area under the curve (AUC) for each patient was calculated as described in Example 1 and the results are shown in Table 3.

TABLE 3
IL-10 response in MS patients at three different doses

| | AUC* ratio | AUC ratio | AUC ratio |
|---|---|---|---|
| Patient No. | (0.2 mg) | (0.6 mg) | (1.8 mg) |
| 101, 201, 301 | 1.04 | 1.26 | 1.67 |
| 102, 202, 302 | 0.99 | 1.08 | 1.87 |
| 103, 203, 303 | 0.96 | 0.99 | 1.85 |
| 104, 204, 304 | 1.74 | 0.86 | 2.37 |
| 105, 205, 305 | 1.28 | 1.86 | 0.97 |

From this table, it can be seen that, first, the highest IL-10 responses are in the range of about 1.5 and higher, that is about 50% higher than baseline. These responses are seen at the highest dose in four of the five patients and in one patient each at the lower two doses, indicating that the highest dose of 1.8 mg daily is a good starting dose for the initial treatment period. Viewed another way, reducing the dose from this level would not be expected to enhance the IL-10 response, and if a patient receiving an initial 1.8 mg/daily dose does not show an IL-10 response of at least about 1.5, the dose should be increased, not decreased in an effort to boost the IL-10 response.

B. IL-10 Response Measured by IL-10 Response/IFN-γ

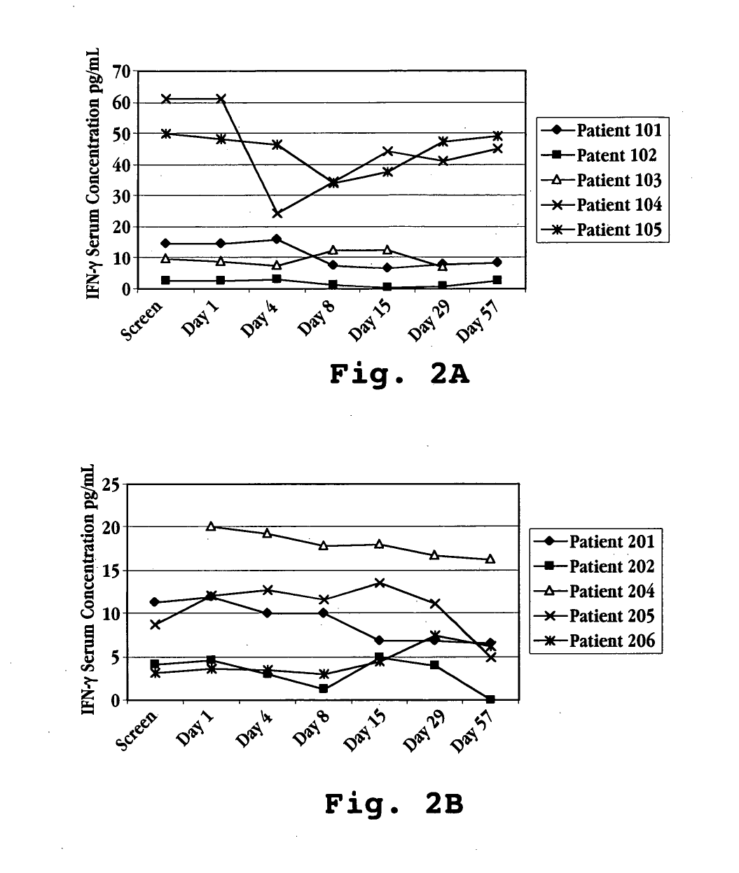

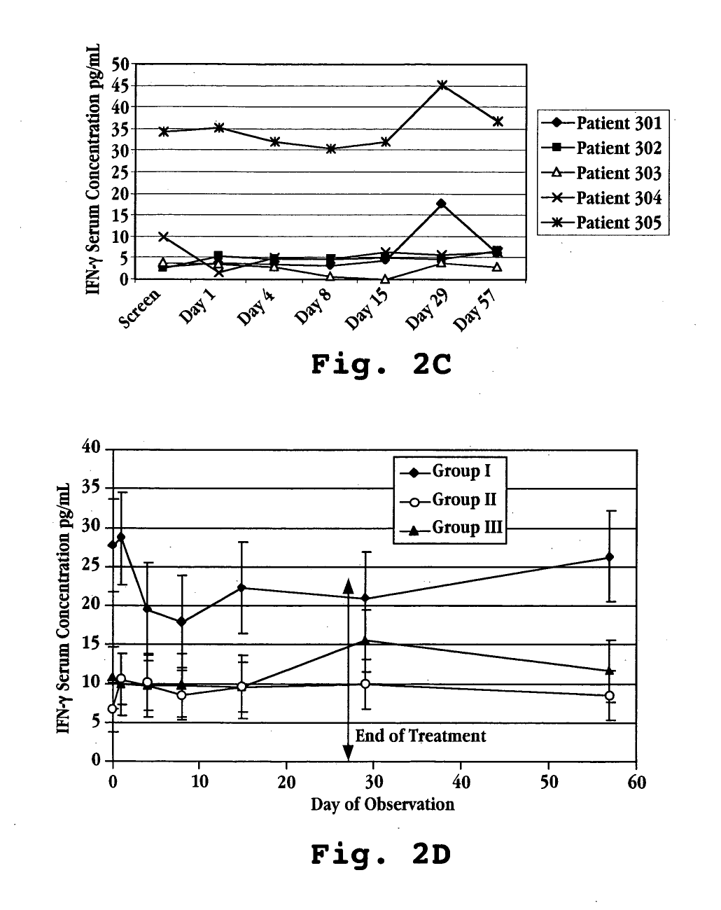

Blood IFN-γ levels were also measured in the same patients, at the same time points, using a commercially available ELISA kits (Genzyme, Cambridge, Mass). The levels of IFN-γ were graphically plotted as shown in FIGS. 2A-2C (IFN-γ) and the area under the curve (AUC) for each patient was calculated as described in Example 1, with the results shown in Table 4.

TABLE 4
IFN-γ response in MS patients at three different doses

| | AUC* ratio | AUC ratio | AUC ratio |
|---|---|---|---|
| Patient No. | (0.2 mg) | (0.6 mg) | (1.8 mg) |
| 101, 201, 301 | 0.60 | 0.72 | 2.44 |
| 102, 202, 302 | 0.47 | 0.89 | 1.88 |
| 103, 203, 303 | 1.05 | 0.89 | 0.44 |
| 104, 204, 304 | 0.66 | 1.42 | 0.57 |
| 105, 205, 305 | 0.82 | 1.54 | 1.02 |

Table 4 shows overall, either a reduction in IFN-γ with IFN-τ administration or no significant change in IFN-γ levels over the initial treatment level.

The use of changes in IFN-γ levels to provide further information on optimal IFN-τ dose can be achieved, for example, by expressing IL-10 response as a ratio of IL-10 response to IFN-γ response, as given in Table 5 below. Using this metric, IL-10 response to the initial treatment does is achieved by an increase in IL-10 response, and this value can be enhanced by a decrease in IFN-γ response.

TABLE 5
IFN-γ response in MS patients at three different doses

| Patient | Dose | IL-10 | | IL-10 response/ |
|---|---|---|---|---|
| No. | (mg) | response | IFN-γ response | IFN-γ response |
| 101 | 0.2 | 1.04 | 0.60 | 1.74 |
| 102 | 0.2 | 0.99 | 0.47 | 2.10 |
| 103 | 0.2 | 0.96 | 1.05 | 0.92 |
| 104 | 0.2 | 1.74 | 0.66 | 2.64 |
| 105 | 0.2 | 1.28 | 0.82 | 1.56 |
| 201 | 0.6 | 1.26 | 0.72 | 1.75 |
| 202 | 0.6 | 1.08 | 0.89 | 1.21 |
| 203 | 0.6 | 0.99 | 0.89 | 1.11 |
| 204 | 0.6 | 0.86 | 1.42 | 0.61 |
| 205 | 0.6 | 1.86 | 1.54 | 1.21 |
| 301 | 1.8 | 1.67 | 2.44 | 0.68 |
| 302 | 1.8 | 1.87 | 1.88 | 1.00 |
| 303 | 1.8 | 1.85 | 0.44 | 4.16 |
| 304 | 1.8 | 2.37 | 0.57 | 4.18 |
| 305 | 1.8 | 0.97 | 1.02 | 0.95 |

### Example 3

### Initial-Treatment IL-10 Level Response in HCV Patients

This example illustrates the application of the method of the invention to patients diagnosed in an active hepatitis C viral (HCV) infection. The dosing schedule for three groups of patients is shown in Table 6.

TABLE 6
Recombinant Ov-IFN-τ Patient Dose Administration

| | | | Volume | | |
|---|---|---|---|---|---|
| | Number | | (mL) | | |
| Dose | of | IFN-τ | per Dose | Total Daily | Total Daily |
| Group | Patients | (mg/mL) | (TID) | Dose (mg) | Dose (U) |
| I | 6 | 1.0 | 0.33 | 1.0 | 1 × 10⁸ |
| II | 6 | 1.0 | 1.0 | 3.0 | 3 × 10⁸ |
| III | 6 | 1.0 | 3.0 | 9.0 | 9 × 10⁸ |

All vials of test material and syringes were kept in a refrigerator maintained at 2 to 8° C. Prior to the self-administration of medication, the patient removed one vial and one syringe from the refrigerator. The cap was removed from the tip of the syringe and the tip of the syringe was placed into the bottle of medication to withdraw the appropriate into the syringe as instructed at the clinic on Day 1.

The tip of the syringe was placed in the mouth and the syringe contents were emptied into the mouth by depressing the plunger. The patient then swallowed the test material. If desired, the patient was allowed to drink a glass of water. The patient noted on his/her diary card the date and time the dose of test material was administered. The above steps were repeated three times per day at approximately eight-hour intervals: once in the morning, once at midday, and once in the evening. Treatment was discontinued at day 85, although IL-10 monitoring was continued until day 169.

A. IL-10 Response Measured by IL-10 AUC/Baseline AUC

Blood samples were taken at defined intervals over a 169 day test period. The samples were analyzed for IL-10 levels in the serum using ELISA kits (Genzyme, Cambridge, Mass.) following the manufacturer's instructions The viral titer of hepatitis C, using reverse-transcriptase polymerase chain reaction, blood levels of 2′,5′-oligoadenylate synthetase (OAS), and the serum concentration of alanine aminotransferase (ALT) were also determined and are not reported here. The levels of IL-10 were graphically plotted as shown in FIGS. 3A-3C, the area under the curve (AUC) for each patient was calculated as described in Example 1 with the results shown in Table 7. As described in Example 1, the AUC calculation was for the period Day 1 to Day 29, that is, the 4-week period starting at the initiation of treatment.

TABLE 7
IL-10 response in HCV patients at three different doses

| Patient | IL-10 response | IL-10 response | II-10 response |
|---|---|---|---|
| No. | (0.33 mg) | (1.0 mg) | (3.0 mg) |
| 1 | 1.19 | 0.72* | 2.53 |
| 2 | 1.32* | 1.30 | 0.95 |
| 3 | 0.91 | 2.72 | 2.62 |
| 4 | 1.40 | 2.53 | 2.34 |
| 5 | 1.23 | 2.17* | 5.07 |
| 6 | 0.92 | 2.51 | 0.93 |

*some data was unavailable

The data indicate that an IL-10 response of at least about 2, i.e., a 100% increase, can be expected in human HCV patients receiving either 3 mg/day or 9 mgs/day IFN-τ, as seen in four of the six patients in each group, and in none of the lowest-dose group (1 mg/day). Thus, the 3 mg/day dose would be a good initial dose, consistent with the data above indicating a preferred starting dose of 1.8 mg/day. If the patients shows a strong IL-10 response, e.g., at least a 100% increase in IL-10 over baseline, the dose could either be maintained over extended treatment, or even reduced somewhat to determine whether the IL-10 response is seen at a somewhat lower dose, e.g., 2 mg/day. The failure of some patients to achieve a good IL-10 response even at the highest doses may be an indication that those patients are less responsive to IFN-τ treatment, and might therefore be placed on a second anti-viral treatment agent.

B. IL-10 Response Measured by IL-10 Response/IFN-γ

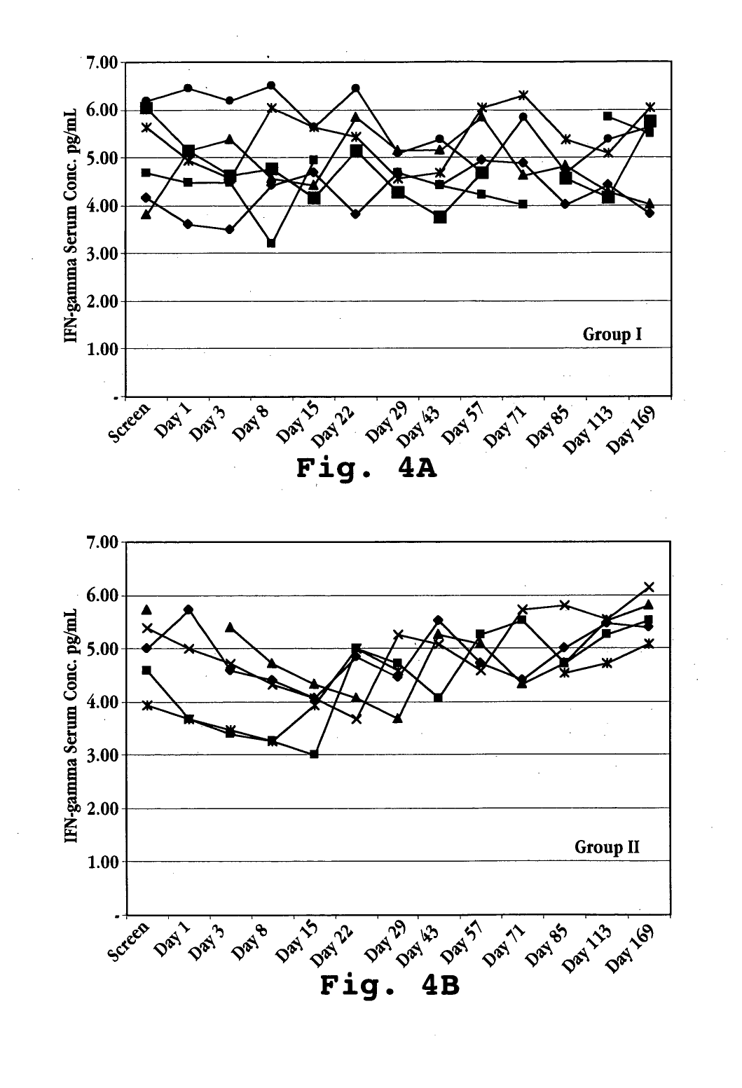

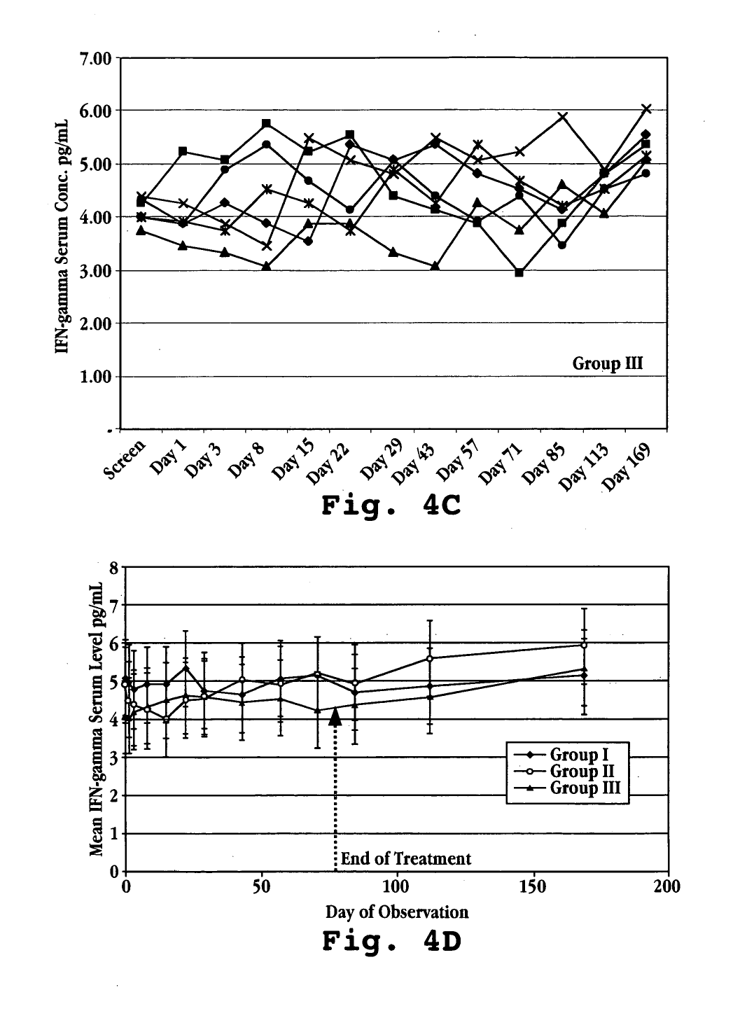

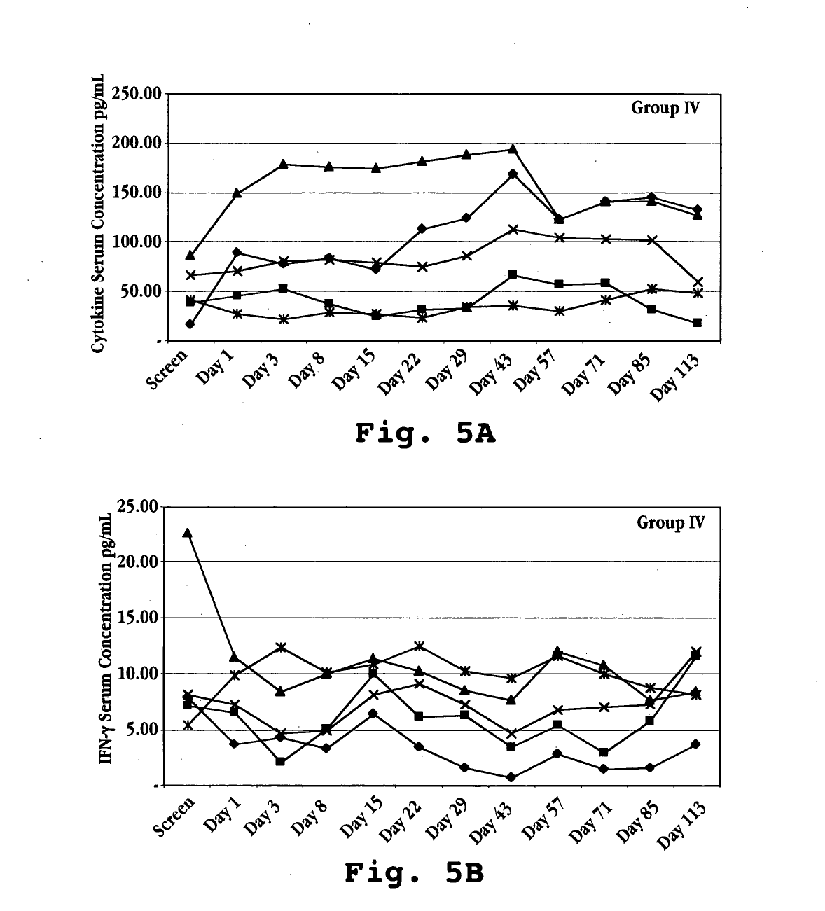

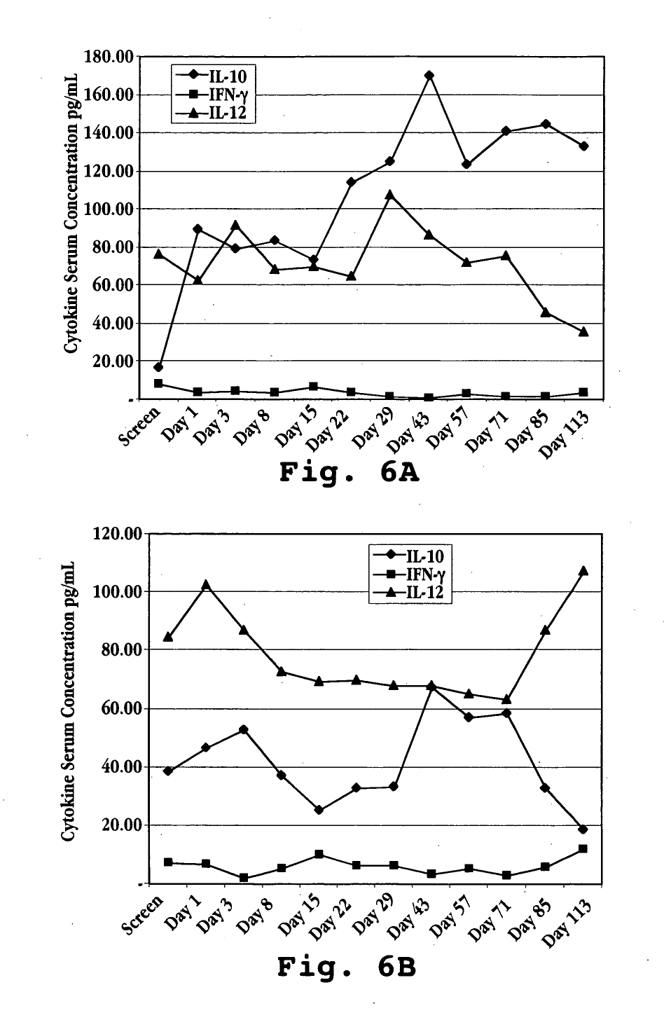

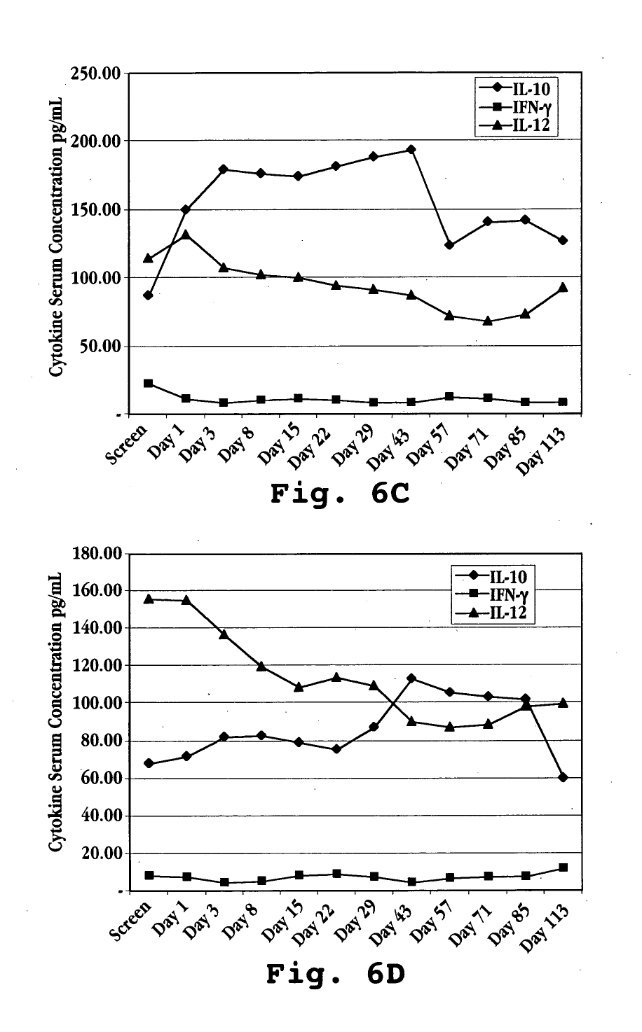

Serum IFN-γ levels were also measured in the same patients, at the same time points, using a commercially available ELISA kits (Genzyme, Cambridge, Mass). The levels of IFN-γ are graphically plotted in FIGS. 4A-4C (IFN-γ) and the area under the curve (AUC) for each patient was calculated as described in Example 1, with the results shown in Table 8.

TABLE 8
IFN-γ response in HCV patients at three different doses

| Patient | IL-10 response | IL-10 response | IL-10 response |
|---|---|---|---|
| No. | (0.33 mg) | (1.0 mg) | (3.0 mg) |
| 1 | 1.02 | —* | 1.09 |
| 2 | 0.95 | 0.90 | 1.24 |
| 3 | 1.32 | 0.84 | 0.95 |
| 4 | 0.77 | 0.79 | 1.05 |
| 5 | 0.95 | 0.79 | 1.05 |
| 6 | 0.98 | 1.04 | 1.09 |

*some data was unavailable

Table 8 shows overall, either a slight increase in IFN-γ with IFN-τ administration or no significant change in IFN-γ levels over the initial treatment level. An-IL-10 response, calculated as the ratio of IL-10 response to IFN-γ, determined from the values in the two table above, is shown in Table 9.

TABLE 9
IL-10/IFN-γ response in HCV patients at three different doses

| Patient | Dose | IL-10 | IFN-γ | IL-10 response/ |
|---|---|---|---|---|
| No. | (mg) | response ratio | response | IFN-γ response |
| 1 | 0.33 | 1.19 | 1.02 | 1.17 |
| 2 | 0.33 | 1.32* | 0.95 | 1.39 |
| 3 | 0.33 | 0.91 | 1.32 | 0.69 |
| 4 | 0.33 | 1.40 | 0.77 | 1.82 |
| 5 | 0.33 | 1.23 | 0.95 | 1.29 |
| 6 | 0.33 | 0.92 | 0.98 | 0.94 |
| 1 | 1.0 | 0.72* | —* | —* |
| 2 | 1.0 | 1.30 | 0.90 | 1.44 |
| 3 | 1.0 | 2.72 | 0.84 | 3.25 |
| 4 | 1.0 | 2.53 | 0.79 | 3.19 |
| 5 | 1.0 | 2.17* | 0.79 | 2.73 |
| 6 | 1.0 | 2.51 | 1.04 | 2.42 |
| 1 | 3.0 | 2.53 | 1.09 | 2.33 |
| 2 | 3.0 | 0.95 | 1.24 | 0.77 |
| 3 | 3.0 | 2.62 | 0.95 | 2.76 |
| 4 | 3.0 | 2.34 | 1.05 | 2.23 |
| 5 | 3.0 | 5.07 | 1.05 | 4.83 |
| 6 | 3.0 | 0.93 | 1.09 | 0.86 |

### Example 4

### Administration of IFNτ Twice Daily to Patients Infected with Hepatitis C

Five patients infected with hepatitis C were recruited for a study. The patients were treated with IFNτ according to the method of Example 3, each patient received 7.5 mg twice daily, for a total daily dose of 15 mg (1.5×10⁹ U). The first dose was taken in the morning, before breakfast. The second dose was taken at least three hours after an evening meal. The treatment period lasted 84 days.

Blood samples were taken at defined intervals over the 113 day test period. The samples were analyzed for IL-10, IL-12, and IFN-γ levels in the serum using commercially available ELISA kits (Genzyme, Cambridge, Mass). The results are shown in FIG. 7A (IL-10), FIG. 7B (IFN-γ), and in FIGS. 8A-8E (IL-10, IL-12, and IFN-γ) for each of the five patients.

TABLE 10
Levels of Interleukin-10, Interferon-γ and their ratios
in patients with hepatitis-C virus treated twice daily under
fasting conditions with 7.5 mg Interferon-τ.

| Patient | IL-10 response | | IL-10 response/ |
|---|---|---|---|
| No. | ratio | IFN-γ response | IFN-γ response |
| 1 | 5.59 | 1.09 | 5.14 |
| 2 | 0.90 | 1.24 | 0.72 |
| 3 | 2.04 | 0.95 | 2.15 |
| 4 | 1.18 | 1.05 | 1.13 |
| 5 | 0.67 | 1.05 | 0.64 |

The data indicate that fasting does not significantly improve the IL-110 response in HCV patients, and may lead to greater variability in the response.

1. In the treatment of a human disease or condition responsive to continued and periodic interferon-T administration in humans, an improvement comprising:
(a) administering to a human subject having such a disease or condition, at each of a plurality of times points over a given time period, a selected, therapeutically indicated amount of interferon-tau,
(b) measuring the level of serum IL-10 in said subject at each of a plurality of time points during the given time period, to determine an IL-10 response in the subject over said time period, and
(c) based on the IL-10 response determined in step (b) adjusting the dose of interferon-tau administered to the human subject, if appropriate, to adjust the subject's IL-10 response in the direction of a desired response to continued interferon-tau administration.

2. The method of claim 1, which further includes repeating steps (a)-(c) at each newly adjusted dose, until a desired IL-10 response is achieved.

3. The method of claim 1, wherein the step (c) is adjusted so as to achieve a patient IL-10 response that is at least 25% above an untreated, baseline IL-10 value determined for the patient.

4. The method of claim 3, wherein the step (c) is adjusted so as to achieve a patient IL-10 response that is at least 50% above an untreated, baseline IL-10 value determined for the patient.

5. The method of claim 3, wherein the step (c) is adjusted so as to achieve a patient IL-10 response that is at least 100% above an untreated, baseline IL-10 value determined for the patient.

6. The method of claim 1, said administering comprises administering ovine interferon-τ or bovine interferon-τ.

7. The method of claim 6 wherein the therapeutically indicated amount of interferon-1 administered in step (a) is at least about 7×10⁶ Units/kg body weight.

8. The method of claim 7 wherein the therapeutically indicated amount of interferon-τ administered in step (a) is at least about 7×10⁷ Units/kg body weight.

9. The method of claim 1, wherein step (a) includes administering to the human subject at each of at least three times points over a given treatment period of at least two weeks.

10. The method of claim 1, wherein step (b) includes determining an IL-10 response level over the treatment period based on an area under the curve calculation.

11. The method of claim 10, wherein step (b) includes measuring the level of serum IL-10 and blood IFN-γ in said subject at each of a plurality of time points during the given time period, and the IL-10 response in the subject is determined in step (b) as a ratio of the IL-10 response to IFN-γ response.

12. The method of claim 10, wherein step (b) includes measuring the level of serum IL-10 and blood IL-12 in said subject at each of a plurality of time points during the given time period, and the IL-10 response the subject is determined in step (b) as a ratio of IL-10 response to IL-12 response.
